(19) United States
(12) Patent Application Publication
(10) Pub. No.: US 2026/0027385 A1
(43) Pub. Date: Jan. 29, 2026

(54) OXYGEN GUIDANCE OF HIGH DOSE RATE BRACHYTHERAPY WITH ELECTRON PARAMAGNETIC RESONANCE

(71) Applicant: THE UNIVERSITY OF CHICAGO, Chicago, IL (US)

(72) Inventors: Bulent Aydogan, Orland Park, IL (US); Boris Epel, Chicago, IL (US); Howard Halpern, Chicago, IL (US); Erik Pearson, Chicago, IL (US); Daniela Olivera Velarde, Chicago, IL (US); Subramanian Sundramoorthy, Chicago, IL (US)

(21) Appl. No.: 19/280,788

(22) Filed: Jul. 25, 2025

Related U.S. Application Data
(60) Provisional application No. 63/675,461, filed on Jul. 25, 2024.

(51) Int. Cl. A61N 5/10; G01D 5/20

(52) U.S. Cl. CPC A61N 5/1071; A61N 5/1027; G01D 5/2013; A61N 2005/1024

(57) ABSTRACT

A system for mapping oxygen levels in tumors includes a needle that is sized to deliver high dose rate brachytherapy to tissue into which the needle is inserted, where the needle includes a radio frequency micro-coil sensor positioned within a shaft of the needle and a spin probe mounted to an outer surface of the shaft of the needle. A processor is in communication with the needle and configured to move the radio frequency micro-coil sensor along the shaft of the needle. The processor is also configured to receive, from the radio frequency micro-coil sensor, oxygen level readings at a plurality of tissue locations along the shaft of the needle. The processor is also configured to generate an oxygen level mapping of the tissue based on the oxygen level readings received from the radio frequency micro-coil sensor.

### CROSS REFERENCE TO RELATED APPLICATIONS

The present application claims priority to U.S. provisional patent application No. 63/675,461 that was filed Jul. 25, 2024, the entire contents of which are incorporated herein by reference.

### BACKGROUND

High dose rate (HDR) brachytherapy is a vital component of cancer treatment due to its precision, effectiveness, and minimal impact on healthy tissues. HDR brachytherapy plays an important role in the comprehensive treatment plans for many cancer patients alongside chemotherapy and external beam radiation therapy (EBRT), offering a chance for tumor control and improved quality of life in a wide range of cancers, including prostate, breast, head and neck, skin, and cervical. Its advantage comes from its capability to precisely deliver a high dose of radiation directly within the tumor site while minimizing exposure to surrounding healthy tissues. This dose conformality is especially valuable in cases where tumors are located near critical organs and when EBRT cannot deliver a curative dose.

### SUMMARY

An illustrative system for mapping oxygen levels in tumors includes a needle that is sized to deliver high dose rate brachytherapy to tissue into which the needle is inserted, where the needle includes a radio frequency micro-coil sensor positioned within a shaft of the needle and a spin probe mounted to an outer surface of the shaft of the needle. A processor is in communication with the needle and configured to move the radio frequency micro-coil sensor along the shaft of the needle. The processor is also configured to receive, from the radio frequency micro-coil sensor, oxygen level readings at a plurality of tissue locations along the shaft of the needle. The processor is also configured to generate an oxygen level mapping of the tissue based on the oxygen level readings received from the radio frequency micro-coil sensor.

The system can also include a radio frequency cable connected to the radio frequency micro-coil sensor and used to move the radio frequency micro-coil sensor along the shaft of the needle. In one embodiment, the spin probe comprises a strip of lithium phthalocyanine (LiPc) mounted to the outer surface of the shaft of the needle. In another embodiment, the needle includes a plurality of strips of LiPc that are mounted lengthwise along the outer surface of the shaft of the needle.

In an illustrative embodiment, the processor is configured to determine a dosage of treatment at each location of the plurality of tissue locations based on the oxygen level reading at each location. The processor is configured to deliver the dosages of treatment through the needle. The dosage of treatment corresponds to the oxygen level at a given tissue location such that the dosage of treatment increases as the oxygen level decreases.

The system can also include a layer of biocompatible oxygen permeable material that encapsulates the spin probe. In one embodiment, the layer of biocompatible oxygen permeable material comprises polydimethylsiloxane (PDMS). In another embodiment, the processor uses an L-band pulse electron paramagnetic resonance (EPR) imaging system to generate the oxygen level mapping. In another embodiment, the radio frequency micro-coil sensor comprises a dielectric core and a slant helix coil mounted on the dielectric core.

An illustrative method for mapping oxygen levels in tumors includes moving, by way of a radio frequency cable, a radio frequency micro-coil sensor along an interior of a shaft of a needle that is sized to deliver high dose rate brachytherapy to tissue into which the needle is inserted. The method also includes receiving, by a processor in communication with the radio frequency micro-coil sensor, oxygen level readings at a plurality of tissue locations along the shaft of the needle. The method further includes generating, by the processor, an oxygen level mapping of the tissue based on the oxygen level readings received from the radio frequency micro-coil sensor.

In an illustrative embodiment, the needle includes a spin probe mounted to an outer surface of the shaft of the needle. In one embodiment, the spin probe comprises a strip of lithium phthalocyanine (LiPc) mounted to the outer surface of the shaft of the needle. The method can also include determining, by the processor, a dosage of treatment at each location of the plurality of tissue locations based on the oxygen level reading at each location. The method can also include delivering, by the processor, the dosages of treatment through the needle. The dosage of treatment corresponds to the oxygen level at a given tissue location such that the dosage of treatment increases as the oxygen level decreases. In another embodiment, a layer of biocompatible oxygen permeable material encapsulates the spin probe. In another embodiment, the method further includes using, by the processor, an L-band pulse electron paramagnetic resonance (EPR) imaging system to generate the oxygen level mapping. In another embodiment, the radio frequency micro-coil sensor comprises a dielectric core and a slant helix coil mounted on the dielectric core.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 is a flow chart that depicts operations performed in an HDR brachytherapy treatment procedure in accordance with an illustrative embodiment.

FIG. 2 depicts results of electron paramagnetic resonance (EPR) oximetry experiments in accordance with an illustrative embodiment.

FIG. 3 shows a large discrepancy of hypoxia readings based on PET (tumor-to-mediastinal ratio (TMR)>1.4) and EPR (pO₂<10) contours in a fibrosarcoma (FSA) tumor in accordance with an illustrative embodiment.

FIG. 4 is a mock cervical plan that illustrates an approach to HDR targeting, where the high-dose area (>200%,) was successfully shifted to a theoretical hypoxic region, minimizing dose to adjacent tissues in accordance with an illustrative embodiment.

FIG. 5 depicts the results of a randomized trial for 3 different tumors in accordance with an illustrative embodiment.

FIG. 6A depicts the experimental setup using a prototype HDR needle with an RFMC in accordance with an illustrative embodiment. The experiment was conducted using a phantom with a controlled gas atmosphere.

FIG. 6B is an EPR amplitude image acquired using the experimental setup of FIG. 6A (slant helix RFMC inside an HDR needle analogous plastic tube with a ring of LiPc on the outer surface) in accordance with an illustrative embodiment.

FIG. 7A provides an overview of the instrumental developments of the proposed system in accordance with an illustrative embodiment.

FIG. 7B is a schematic representation of components of the EPR imaging system in accordance with an illustrative embodiment.

FIG. 7C depicts a spin probe coating in the form of axial strips in accordance with an illustrative embodiment.

FIG. 7D depicts a spin probe coating in the form of rings in accordance with an illustrative embodiment.

FIG. 7E depicts a spin probe coating in the form of a uniform surface coating in accordance with an illustrative embodiment.

FIG. 7F depicts a spin probe coating the form of an array of discrete points in accordance with an illustrative embodiment.

FIG. 8 is a simplified diagram of the L-band system for use in the proposed system in accordance with an illustrative embodiment.

FIG. 9A shows B₁⊥ simulations of a slant helix coil formed at the end of a transmission line conducted using the COMSOL Multiphysics® analysis software in accordance with an illustrative embodiment.

FIG. 9B is a rendering of a radio frequency micro-coil in accordance with an illustrative embodiment.

FIG. 10 depicts the layout of a test chamber for validating the RFMC in accordance with an illustrative embodiment.

FIG. 11 is a table that depicts cable design requirements for the autosequencer in accordance with an illustrative embodiment.

FIG. 12 depicts a computing system for measuring and mapping oxygen levels of tumors in accordance with an illustrative embodiment.

### DETAILED DESCRIPTION

High dose rate (HDR) brachytherapy is a procedure that can be used to help treat various types of cancer including prostate, breast, head and neck, skin, cervical, etc. In traditional HDR brachytherapy, multiple plastic needles are inserted into a tumor to deliver brachytherapy to the patient using a radioisotope (e.g., Iridium-192). Current treatments typically employ an average of 20 HDR needles, with the option to increase the quantity as needed. The procedure typically starts at least a week before the first treatment with both diagnostic magnetic resonance imaging (MRI) and computed tomography (CT) imaging sessions for pretreatment planning. The pretreatment planning includes determination of an optimal placement of needles to cover the entire tumor. The objective is to ensure conformation between the administered radiation dose and the spatial dimensions of the tumor. On the day of treatment, the patient is taken to the operating room, and a spinal block or general anesthesia is administered. The physician inserts a predetermined number of thin, hollow plastic needles into the tumor under ultrasound or x-ray guidance to achieve the planned needle arrangement. The patient is returned to the radiation oncology department, and a planning CT scan is acquired. Treatment planning begins with digitally identifying the needle tracks using the images followed by dose planning, and this process takes 2-4 hours depending on the treatment's complexity.

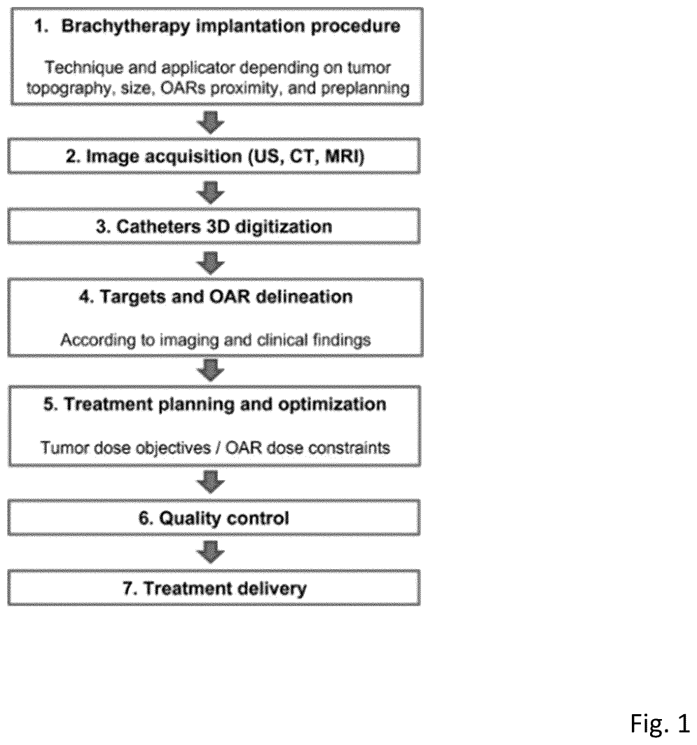

FIG. 1 is a flow chart that depicts operations performed in an HDR brachytherapy treatment procedure in accordance with an illustrative embodiment. A brachytherapy implantation procedure is performed, in which the technique and applicator depend on the tumor size, topography, the proximity of organs at risk (OAR), preplanning, etc. Image acquisitions are obtained using any type of imaging procedure such as MRI, CT, ultrasound (US), etc. Catheter three-dimensional (3D) digitization is performed, along with the delineation of target tumor(s) and organs at risk. The delineation can be performed according to the imaging and clinical findings. Treatment planning and optimization is performed to determine tumor dose objectives and/or OAR dose constraints. Quality control is conducted, followed by delivery of the brachytherapy treatment. Due to the challenges discussed below, traditional HDR brachytherapy is generally conducted without consideration of oxygen levels in the tumor tissue.

It has been found that a lack of oxygen (O₂), or hypoxia, is a nearly universal characteristic feature of solid tumors due to uncontrolled cell proliferation, altered metabolism, and their inadequate and heterogeneous vascular network. The lack of oxygen is recognized to be a primary cause of poor clinical outcomes due to the adaptations of the tumor to hypoxic conditions resulting in resistance to all known therapy modalities including radiotherapy, chemotherapy, and immunotherapy.

Hypoxia in solid malignancies is a critical parameter affecting clinical outcomes, particularly for radiation therapy (RT). The efficacy of RT can be predicted more significantly by O₂ levels in the tumor than by stage, tumor morphology, or tumor size. Monitoring of tumor O₂ has been shown to have very significant implications for improving decision-making throughout cancer therapy, from the time of initial diagnosis, during primary treatment, and reconstructive procedures. Heterogeneous regions of tumor hypoxia are particularly resistant to conventional treatments, reducing patient tumor control rates substantially in most solid tumors (e.g., notably in the prostate, brain, head and neck, pancreas, lung, and cervix). In cervical cancer patients, for example, the 6-year overall survival decreases from 87% to 29% with hypoxic tumors having less than 10 torr pO₂. Moreover, approximately 48% of all cervical patients suffer from hypoxic radioresistant tumors. Similarly, it has been shown that 8-year freedom from biochemical failure in prostate cancers drops from 78% to 46% in patients with hypoxic tumors. However, this knowledge has not yet resulted in any impact on actual clinical practice or patient results. One plausible explanation for this is the lack of means to measure temporal and spatial changes of hypoxia accurately in a manner that can guide RT and/or monitor effect of O₂ interventions.

Enhancing tumor oxygenation has long been a focus of active research aimed at overcoming treatment resistance. However, the absence of repeated, rapid, and precise O₂ measurements with sufficient temporal resolution has significantly limited progress in this field. Clinical trials to test the efficacy of O₂-enhancing interventions have not achieved practice-changing results. In hindsight, this was primarily due to an inability to identify which patients had tumors that responded to O₂-enhancing strategies and to monitor the time course of these responses, which is crucial for optimizing radiation delivery scheduling. Recent EPR oximetry findings highlight the importance of repeated and direct measurements of O₂ for optimizing O₂-enhancing interventions.

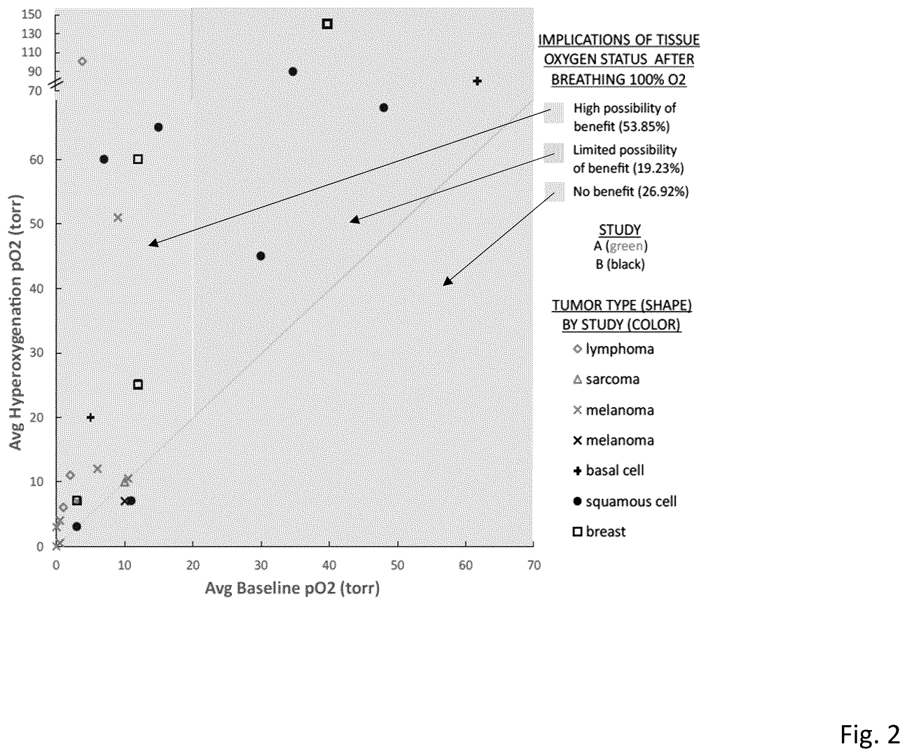

FIG. 2 depicts results of electron paramagnetic resonance (EPR) oximetry experiments in accordance with an illustrative embodiment. Specifically, FIG. 2 shows the results of EPR measurements conducted on 25 patients with superficial tumors (<11 millimeter (mm) depth), and illustrates that not all patients respond to O₂ enhancing interventions. These experiments show that about half of the tumors are unlikely to show significant changes in radiation response due to interventions such as breathing O₂ rich air or carbogen, either because they did not respond to intervention or already had high O₂ levels, making them less resistant to radiation from hypoxia. Failing to account for these variations could undermine the integrity of clinical trial designs. Additionally, interest in overcoming tumor hypoxia has surged in recent years leading to rapid advances in methods such as MnO₂ nanoparticle pharmaceuticals and ultrasound sensitive O₂ microbubbles. These emerging approaches hold promise for addressing hypoxia but require dynamic, real-time monitoring of tumor O₂ levels to evaluate and further maximize their clinical effectiveness.

Electron paramagnetic resonance oximetry utilizes signals from unpaired electron spins of a stable, paramagnetic O₂-sensing material (aka spin probe), providing information about the local O₂ levels, pH, thiol concentration, and tissue redox state. Spin probes, whose relaxation rates are proportionately impacted by molecular O₂, are used because molecular O₂ is extremely hard to detect on its own. Preclinical EPR imaging based on water-soluble spin probes provides absolute O₂ maps in tumors of mice and rabbits. EPR O₂ imaging accurately measures local pO₂ levels in mammalian tumor tissues, as confirmed by stereotactic OxyLite measurements, the gold standard for measuring tissue pO₂, via fiberoptic phosphorescence quenching. While clinical EPR imaging is under development, single point EPR oximetry measurement has been used in more than 100 patients to date, including 35 patients with an OxyChip implanted in the tumor with a minimally invasive procedure. This single-point EPR oximetry method, was thought to be extendable to readings in 4 single points with additional coils and sensing beads, nevertheless further improvement proved to be clinically challenging. This method affords fast, highly reproducible, and precise O₂ readings but is limited to a few points and less than 1 centimeter (cm) depth in patients.

A survey of existing clinical oxygen imaging technologies and their limitations is discussed below. Currently, no clinical O₂ imaging methods are both noninvasive and capable of absolute, serial imaging with sufficient spatiotemporal resolution to capture O₂ dynamics in a tumor, which is critical for oxygen-guided treatments and evaluating interventions that increase tumor O₂ levels. While clinical options like 18F-misonidazole, positron emission tomography (PET), and MRI provide some O₂-related information, they neither measure O₂ directly in tumors and surrounding tissues (e.g., BOLD MRI measures unsaturated hemoglobin in blood vessels), nor do they allow repeated, same-site measurements to track hypoxia over time. Both are limited by long acquisition times, making them impractical for real-time treatment planning or assessing intervention efficacy.

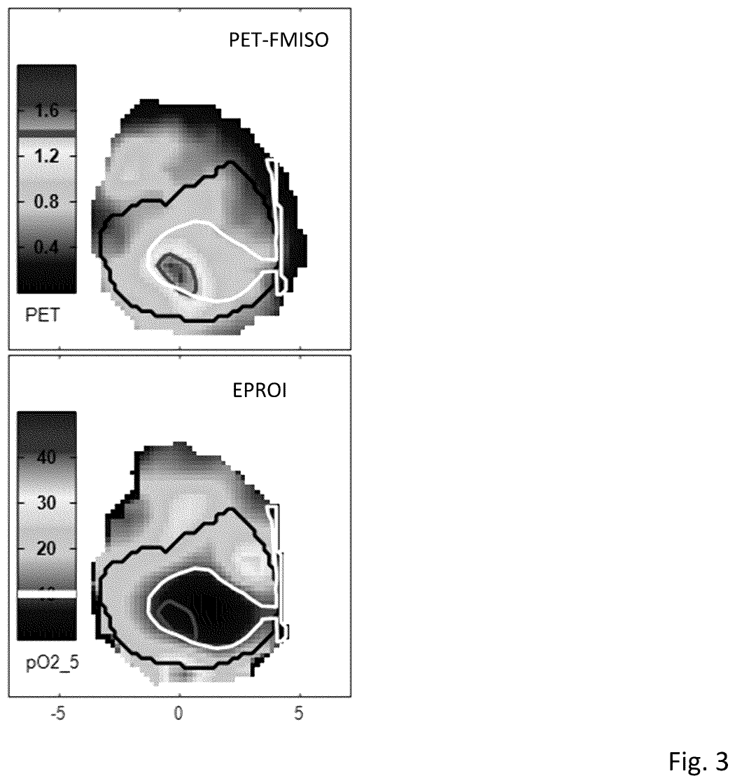

Furthermore, the inventors have revealed considerable discrepancies between PET O₂ imaging and EPR measurements, emphasizing the need for reliable, direct O₂ assessment. This finding resulted in research to enhance PET hypoxia imaging with corrections based on EPR. PET hypoxia is dependent on tumor vascularization. As such it is only a surrogate, and not a measure of absolute O₂. FIG. 3 shows a large discrepancy of hypoxia readings based on PET (tumor-to-mediastinal ratio (TMR)>1.4) and EPR (pO₂<10) contours in a fibrosarcoma (FSA) tumor in accordance with an illustrative embodiment. FIG. 3 reveals an underestimation of tumor hypoxia by PET, which is a factor in the limited success of dose painting strategies based on PET imaging. In summary, while PET and BOLD MRI reflect factors related to tumor O₂, they do not reliably and directly measure actual O₂ levels or capture dynamic changes, making them unsuitable for addressing hypoxia. This validation gap can be addressed by clinical single-point EPR oximetry.

An alternative method for measuring tissue oxygenation for HDR is currently in development. This approach uses silicone and silicone oils which have O₂-dependent changes in MR T₁ relaxation times. However, the MR T₁ changes are relatively small resulting in signal-to-noise ratio (SNR) challenges. Also, reported results only differentiate between 0% and 21% (0 and 159.6 torr pO₂) O₂ level, and this method requires a minimum of 30 minutes of expensive and often not readily available MR scanner time.

Other clinically relevant oxygen measurement techniques include a variety of optical methods such as near-infrared spectroscopy (NIRS) and fluorescence quenching (FQ). While in principle FQ can be applied for brachytherapy and is used here for single-point validation, achieving the goals of this system with FQ would present a rather formidable engineering task due to the necessity of forming multiple optical paths inside the HDR needle The optical fiber robustness of this approach is also questionable.

Clinical implementation strategies and clinical feasibility of the proposed system is discussed below. The proposed innovative development of O₂ imaging technology will, for the first time enable treatment personalization strategies to address hypoxia properly such as: (1) identifying patients responsive to O₂-enhancing interventions through serial measurement of tumor oxygenation and response to hyperoxygenation; (2) optimizing treatment timing to align with peak tumor oxygenation levels for maximal therapeutic benefit; and (3) targeting higher HDR doses specifically to hypoxic regions (OGHDR), an approach involving dose painting to intensify treatment in hypoxic tumor areas while reducing doses in well-oxygenated regions, enabling personalized treatment.

Typically, HDR brachytherapy is done after an external beam procedure, resulting in reduction of tumor bulk. The residual tumor is more likely to contain hypoxic, radioresistant cells, and as such O₂ monitoring at the time of HDR brachytherapy (after

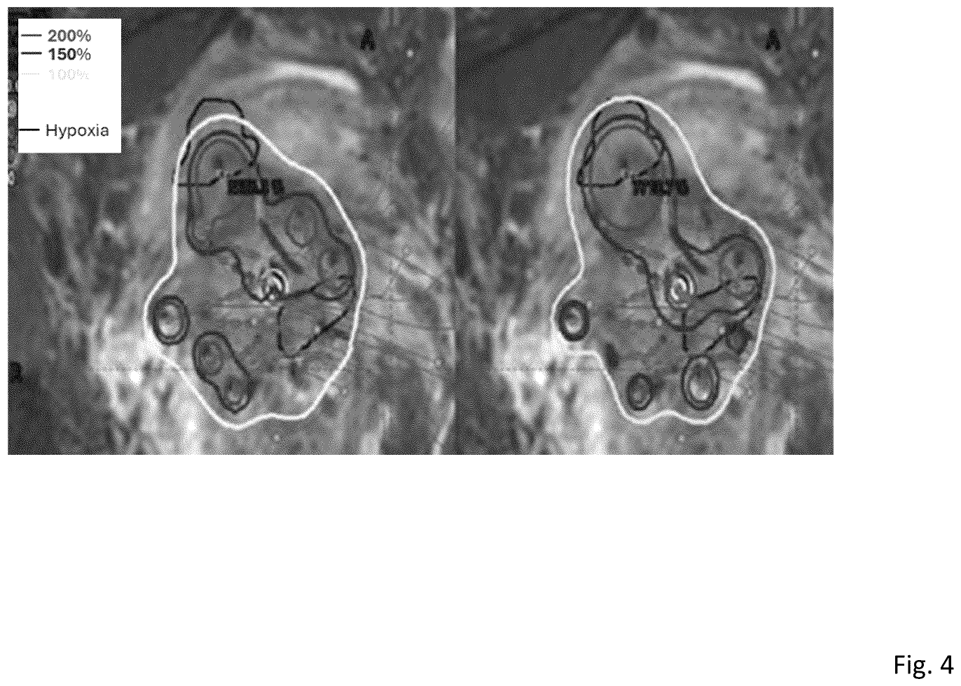

EBRT) is particularly relevant. HDR brachytherapy often results in localized regions receiving doses as high as 250% or more of the prescribed dose. With OGHDR, these high-dose regions can be targeted to hypoxic areas, reducing exposure to surrounding tissues. FIG. 4 is a mock cervical plan that illustrates an approach to HDR targeting, where the high-dose area (>200%,) was successfully shifted to a theoretical hypoxic region, minimizing dose to adjacent tissues in accordance with an illustrative embodiment. Specifically, the left portion of FIG. 4 shows an original plan without oxygen guidance. The right side of FIG. 4 shows an oxygen-guided plan with optimized 100% dose coverage to the anterior hypoxic region and repositioned doses>150% to the left posterior hypoxic area. Hot spots on both sides were reduced, decreasing dose to surrounding healthy tissue. A future clinical trial that integrates these strategies—enabled uniquely by the proposed technology—holds the potential to significantly improve patient outcomes while advancing the understanding of the dynamic and molecular changes associated with clinical hypoxia.

Described herein is a first-in-kind technology combining HDR brachytherapy treatment with EPR oximetry enabling personalized OGHDR. The primary challenges associated with single point EPR oximetry lie in its invasive nature, limited number of samples (<4), and depth of measurement (<1 cm). However, multiple HDR brachytherapy needles inserted into the tissue provide a direct conduit for concurrent and continuous O₂ assessment of the tumor. This synergy between HDR and EPR not only addresses the concerns around invasiveness with dedicated EPR implants but also introduces an opportunity for volumetric O₂ monitoring.

When an EPR sensing material (e.g., a spin probe), that is O₂-equilibrated with the surrounding tissues, is incorporated on the outer surface of an HDR needle, a rapid reading of the EPR signal becomes possible. Thus, by incorporating a fast-moving signal detector (e.g., a radio frequency microcontroller (RFMC)) within the needle, real-time readings can be obtained, enabling O₂ imaging along the HDR needle trajectory. Given the multiple HDR needles distributed throughout the tumor and the potential for increased numbers to achieve higher resolution, this approach enables the generation of a sparse 3D image depicting the spatial distribution of O₂. This process presents a significant technological leap from singlepoint O₂ measurement towards the first clinically relevant volumetric O₂ measurements needed for personalized radiotherapy. This groundbreaking technology meets the essential clinical requirements by enabling the evaluation of spatial and temporal changes in tumor O₂ levels during both therapy and intervention methods aimed at increasing tumor O₂ levels. This enables multiple routes of treatment personalization such as intensifying doses to hypoxic regions and/or synchronizing treatment delivery with the peak tumor O₂ levels after patients breathe O₂-rich air.

Five technological components of the proposed system are: (1) O₂-sensing HDR needles featuring a LiPc (Lithium Phthalocyanine) spin probe integrated onto the outer surface, encapsulated in a biocompatible O₂ permeable polymer, such as polydimethylsiloxane (PDMS); (2) Scalable production of LiPc for widespread commercial availability; (3) An RFMC and cable designed to navigate within plastic HDR needles for evaluating tissue O₂ levels; (4) An L-band pulse EPR imager encompassing a magnet, gradients, and pulsed acquisition hardware; and (5) An autosequencer to automate fast EPR O₂ imaging through HDR needles. The ultimate vision for the proposed technology is an independent device, matching the form factor of an HDR afterloader, that enables repeated O₂ measurements both inside and outside the HDR suite, during or between treatments, with straightforward operation for clinicians already familiar with the afterloader.

Key strengths of the technology are rapid, direct, and low-cost measurement of tissue O₂ for any clinical sites treated by HDR brachytherapy without added extra invasiveness. The proposed instrumentation is highly compatible with existing clinical workflows, thanks to its compact design, which allows for easy integration into most radiation oncology departments. With efficient measurement capabilities and a user-friendly interface, the system can be designed to be operated by current staff without the need for extensive training. Furthermore, the proposed technology has a modest upfront system cost compared with PET and MR systems, along with lower operational expenses.

This technology eliminates the requirement for contrast agents or radiotracers with limited shelf-life and special shipping and handling considerations, further contributing to its cost-effectiveness and case of use. The proposed design also offers multiple routes for measurement parallelization (potential for multiple needles, multiple coils, multiple dwell positions, etc.), resulting in denser sampling, faster measurement and/or expanded coverage (larger field-of-view). Furthermore, additional needles may be used for simultaneous monitoring during radiation delivery. The proposed technology enables highly personalized treatment planning guidance and scientific opportunities to investigate practical and effective methods for combatting hypoxia and improving outcomes.

The proposed system has provided the first mammalian preclinical evidence that localizing high dose radiation to hypoxic regions can improve outcomes. The approach used conformal radiation blocks with focused boost radiation on either hypoxic or well oxygenated tumor areas to test the hypothesis that treating hypoxic (pO₂≤10 torr) subregions of a tumor defined by EPR imaging with a higher dose may improve outcomes. The three syngeneic tumor models used were MCa-4 mammary adenocarcinomas, SCC7 squamous cell carcinomas, and FSa fibrosarcoma in the legs of C3H mice.

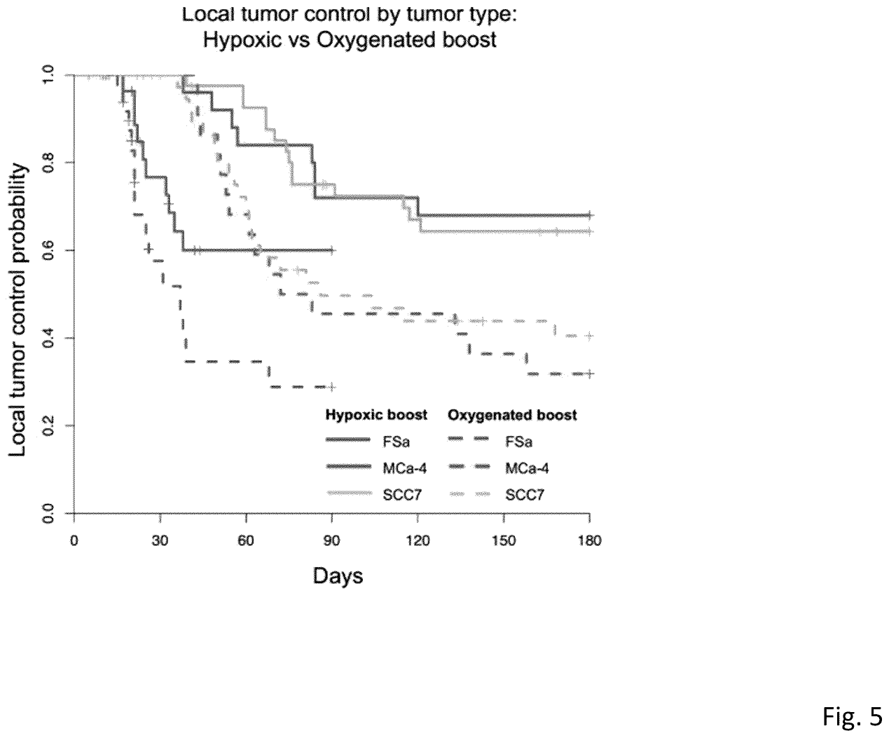

The whole tumor first received TCD₂₀, the dose level to achieve control in 20% of tumors. Each animal then received an additional radiation boost of 13 Gy (additional dose to achieve TCD₉₀) randomly assigned to either all hypoxic subregions of a tumor (‘hypoxic boost’) or to well-oxygenated regions of roughly equal volume (‘oxygenated boost’). Thus, the only difference between tumor treatments was the boost location determined from the EPR imaging. It was found that the addition of the 13 Gy boost dose to hypoxic tumor regions significantly enhanced outcome (two folds improvement) in all three cell lines compared to the control (oxygenated boost). FIG. 5 depicts the results of a randomized trial for 3 different tumors in accordance with an illustrative embodiment. In FIG. 5, the solid line (boosting hypoxic volume (pO₂≤10 torr) and dotted line (boosting comparable volume of normoxic region) illustrate that hypoxic boosting significantly (p<0.001) improves survival. This data from the trial group provides the first preclinical evidence for the advantage of boosting hypoxic subregions in tumors during HDR brachytherapy. This underscores the critical importance of accurately identifying hypoxic regions, addressing a key gap in understanding and managing tumor hypoxia.

Scalable chemical spin probe synthesis is discussed below. EPR measurements involve the use of an exogenous spin probe. The inventors used LiPc as the spin probe for its superior properties for the proposed development. The LiPc spin probe comes in three polymorphs: α, β, X, amongst which only the X phase is O₂ sensitive because of channel configuration in the three-dimensional structure. The standard LiPc electrochemical synthesis has proven difficult to replicate due to the nuances of the probe deposition on the working electrode resulting in significant batch-to-batch variations. Furthermore, it has serious scaling issues typically limiting batch sizes to less than one gram.

Therefore, for the eventual commercialization of the proposed technology, it was important to develop a scalable method to produce quality LiPc in larger quantities and with reproducible properties. The inventors developed a novel chemical synthesis method using a wet chemical route in which the precursor solution is placed in a larger vessel containing the oxidant which reacts via slow vapor diffusion. This chemical route enables complete phase control by changing the oxidant choice and precursor (Li₂Pc) concentration, which have been tuned to obtain the desired pure X phase, evaluated via X-ray Diffraction (XRD) analysis. The resultant product can be easily filtered and the whole process is readily scalable and does not require expensive platinum electrodes, unlike the original method. The inventors successfully scaled up the production and verified quality production with the batch-to-batch comparison with increased EPR signal strength. This development ensures a ready supply of an important component for the system.

With respect to RFMC design, the inventors have made and tested an implantable resonator design consisting of multiple micro helical coils that greatly enhanced the sensitivity of the CW EPR measurements using an LiPc spin probe. The new design was optimized using finite element analysis (COMSOL Multiphysics®, COMSOL AB, Stockholm Sweden) and implemented in hardware. The improvements enabled O₂ measurements with an SNR increase from 17 to 375, more than 20-fold, in a tissue-mimicking phantom even at 23 cm depth. Unlike earlier designs which were limited to <1 cm depth of measurement, the solution proves capable of reaching the depths necessary to target human tumors at any depth.

To demonstrate feasibility of using HDR needles for O₂ imaging, the inventors have performed EPR imaging with a prototype version of the design. A marginally oversized version of an HDR needle was fabricated from a polymer tube with two shallow rings machined in the outer surface, which were filled with an LiPc/PDMS (polydimethylsiloxane) mixture. A 5-turn slant helix RFMC was hand wound, the resonant frequency was tuned with minute shape adjustments, and it was attached to a UT-047 coaxial cable. The system was set up in a 720 MHz pulse EPR system for small animal imaging with the RFMC inside the HDR needle analog. The coil was able to produce 60 nanosecond (ns) π/2 pulses with 48 dBm of power and produce an EPR amplitude image.

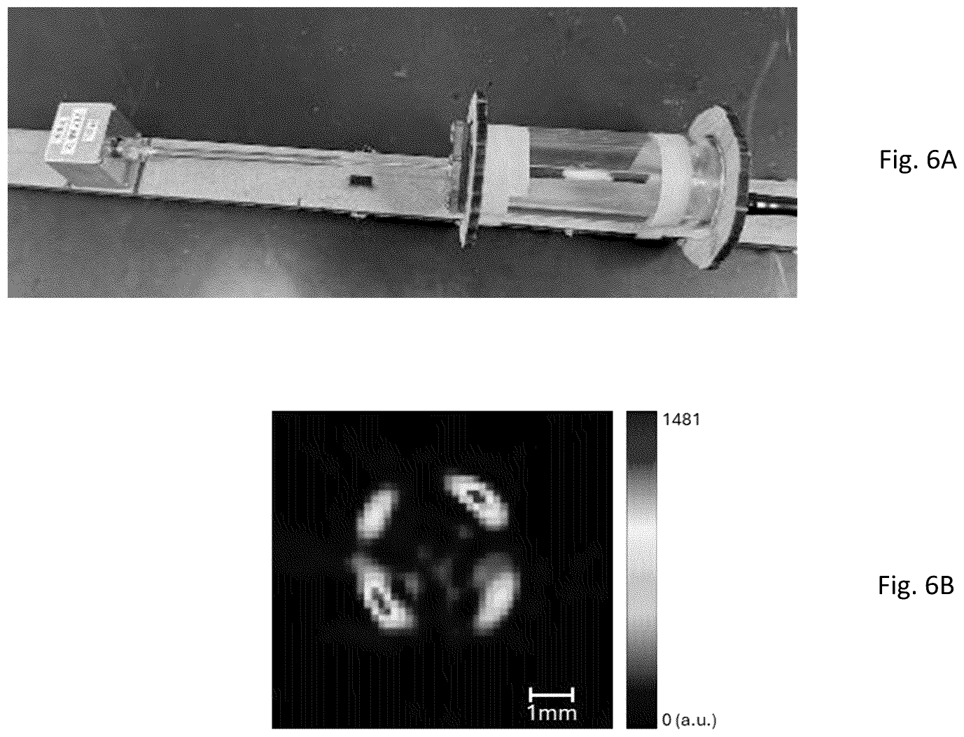

FIG. 6A depicts the experimental setup using a prototype HDR needle with an RFMC in accordance with an illustrative embodiment. The experiment was conducted using a phantom with a controlled gas atmosphere. FIG. 6B is an EPR amplitude image acquired using the experimental setup of FIG. 6A (slant helix RFMC inside an HDR needle analogous plastic tube with a ring of LiPc on the outer surface) in accordance with an illustrative embodiment. The four lobe amplitude pattern shown in FIG. 6B is expected and due to the distribution of magnetic fields in the slant helix RFMC. The image demonstrates strong signal presented by arbitrary unit (au) ranging 0 to 1481 from the LiPc, at the surface of the plastic needle demonstrating the capability of EPR imaging of O₂.

Initial tests demonstrated that coupling above 40 decibels (dB) is achievable with a 30 cm transmission cable. Notably, signal strength remains stable even with variations in cable length from the coupling unit, a critical requirement for HDR applications. This confirms the system capability to produce O₂ images in an HDR compatible configuration. This comprehensive proof of concept demonstrates the resources and capabilities of the proposed system.

With respect to system components and safety, the system can utilize a low-cost EPR magnet (e.g., by Clin EPR, LLC), which is classified as a non-significant risk device due to its low magnetic field, requiring no further development and eliminating the need for a specialized room like MRI. The autosequencer can be based on the Varian's Bravos HDR afterloader, including human factors and safety considerations. The EPR bridge added to the afterloader can be electrically isolated for patient safety and the RF emissions are low powered on the scale of other medical devices. In one embodiment, the HDR needles can be derived from the Varian needles in current clinical use with the addition of an EPR spin probe encapsulated in biocompatible polymers. The materials used can be similar to those used in the IDE-approved OxyChip (G130260).

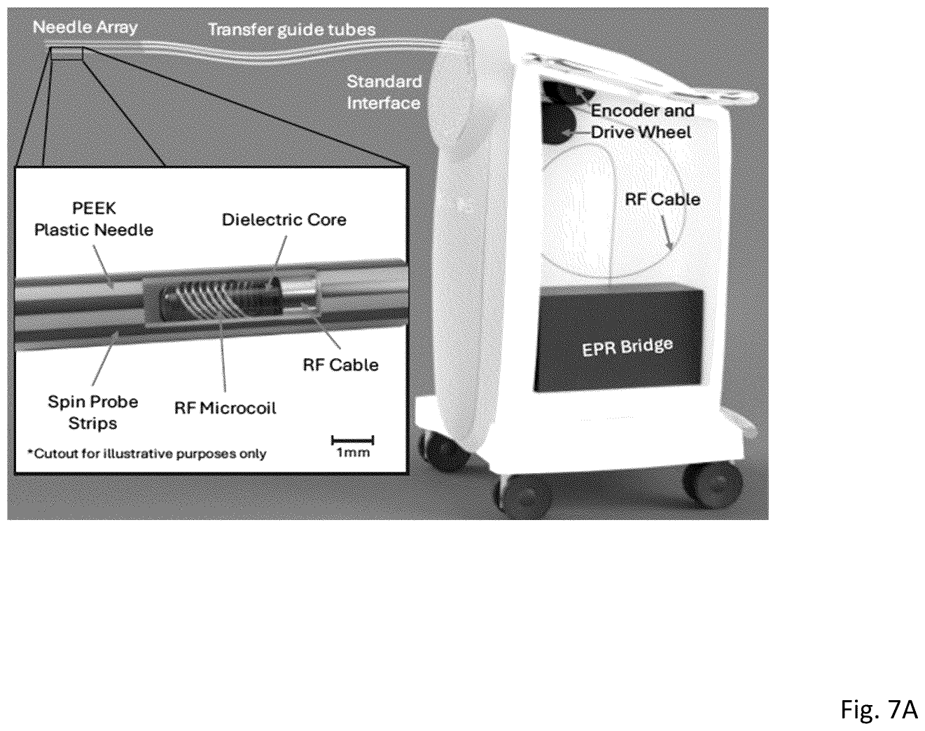

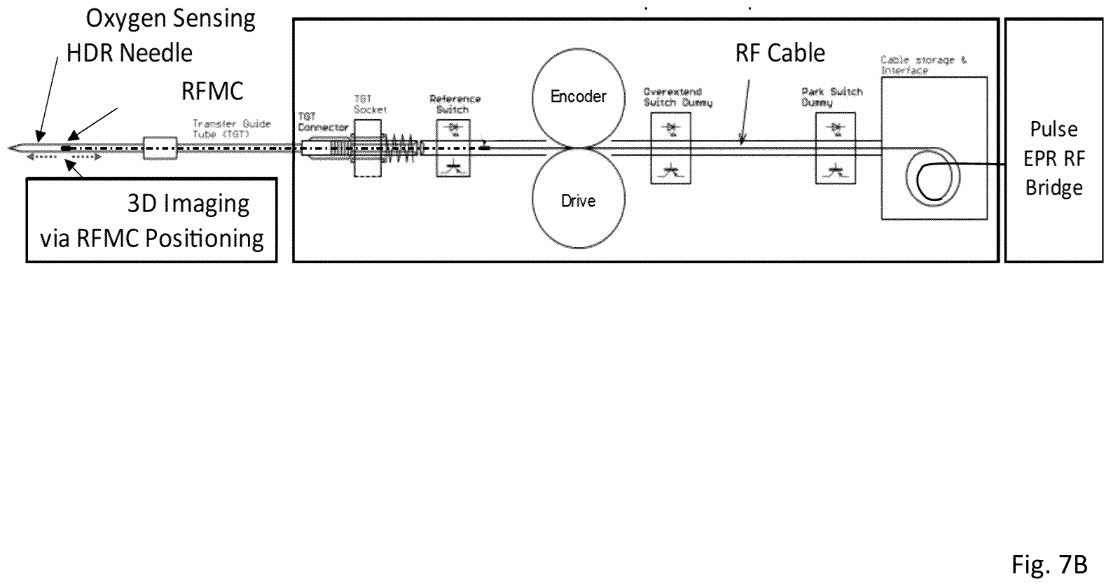

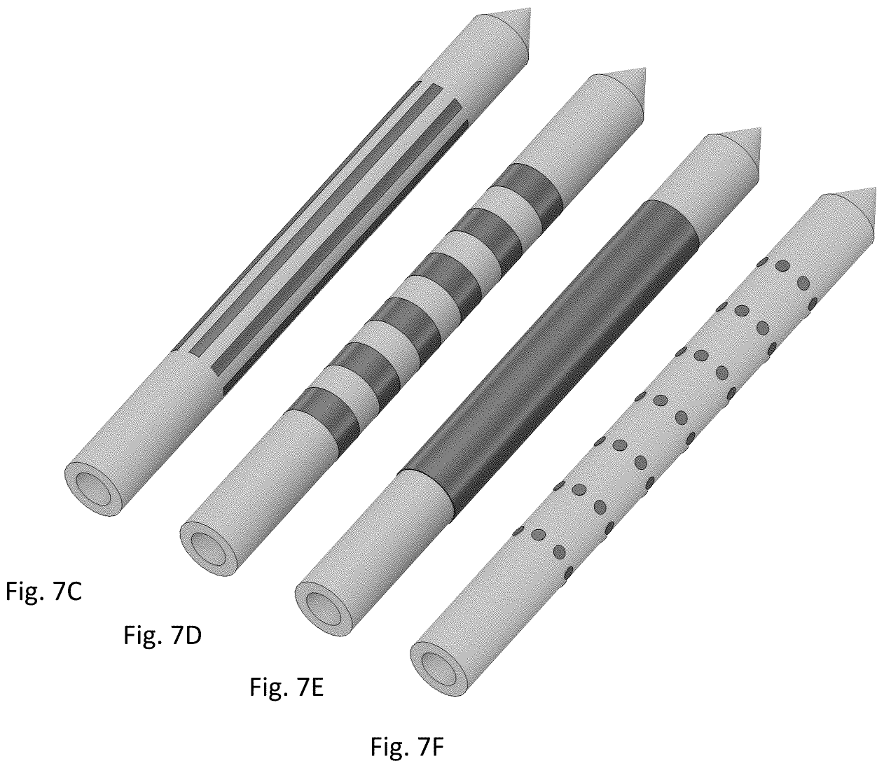

FIG. 7A provides an overview of the instrumental developments of the proposed system in accordance with an illustrative embodiment. As shown, in this embodiment the system includes an L-band pulse EPR O₂ imaging system including an EPR Bridge, an EPR sensor (RFMC) connected with an RF cable to be deployed into a specially designed O₂ sensing HDR needle. The O₂-sensitive HDR needle is embedded with an LiPc paramagnetic probe. The system also includes a benchtop prototype autosequencer, which can be functionally equivalent to a Varian autoloader. In one embodiment, the EPR technology can be shaped into a brachytherapy-compatible format by attaching the RFMC to one end of a coaxial cable serving both as a signal conduit and mechanical element for positioning controlled by the autosequencer. The other end of the cable will be connected to the RF bridge.

FIG. 7B is a schematic representation of components of the EPR imaging system in accordance with an illustrative embodiment. As shown, the system includes an oxygen sensing HDR needle, an RFMC embedded within the needle, an autosequencer for controlling RFMC position in the HDR needle, and a pulse EPR RF bridge. In alternative embodiments, the system can include fewer, additional, and/or different elements. FIGS. 7C-7F depict visual representations of different configurations of the spin probe coating on the needle. FIG. 7C depicts a spin probe coating in the form of axial strips in accordance with an illustrative embodiment. FIG. 7D depicts a spin probe coating in the form of rings in accordance with an illustrative embodiment. FIG. 7E depicts a spin probe coating in the form of a uniform surface coating in accordance with an illustrative embodiment. FIG. 7F depicts a spin probe coating the form of an array of discrete points in accordance with an illustrative embodiment. In the embodiments of FIGS. 7C-7F, the count, spacing, and size of the spin probe coating all are variables to be optimized for SNR, manufacturability, and mechanical strength. In alternative embodiments, different configurations/patterns of the spin probe may be used.

In the embodiment of FIG. 7A, the system includes a mobile base that allows the system to be moved out of the way for rapid transition from oxygen measurement to treatment using Bravos. Also, without the need for shielded isotope storage, there is ample space to integrate the EPR Bridge, and the existing hardware for precise radioisotope positioning can be replicated to position the EPR sensor. In one embodiment, design criteria of the proposed system includes (A) O₂-sensitive HDR needles fully compatible with conventional HDR needles (mechanical properties, dimensions, interface, etc.); (B) Positional spatial resolution of ≤2 mm. (C) EPR hardware operating at 1.2 GHz; (D) Pulse EPR operational mode, electron spin echo (ESE) O₂ measuring pulse sequence and (E) Imaging time—1.5 sec/point. In alternative implementations, different design criteria may be used.

As discussed, the proposed system includes an O₂ imager for clinical HDR brachytherapy. The EPR imaging system has been designed to provide accurate maps of O₂ levels with high sensitivity, high absolute accuracy within 1 torr, and high spatial accuracy (˜1 mm). Design of the system included development of an L-band pulse EPR system for fast and robust oxygen measurement. Two major acquisition techniques used for EPR are pulse and continuous wave (CW). Pulse EPR can be applied to a limited scope of applications and spin probes, but within that scope, it is exceptionally successful. Most prior work with particulate spin probes was performed using CW. However, with the increased availability of pulse in vivo instruments, more and more studies are performed with pulse EPR. The inventors demonstrated that pulse EPR O₂ imaging using soluble trityl spin probes has a massive advantage over CW both in terms of precision and absolute accuracy.

Pulse EPR's main advantages over CW are: (1) Simplicity of hardware. A lower requirement on the magnetic field (B₀/B₁) homogeneity, no modulation field, and simpler RF bridge design. Pulse EPR low-Q resonators can be tuned during fabrication and do not require re-tuning and re-matching for each use; (2) Richness of methodology. Pulse EPR enables direct measurement of T₁ and T₂, where 1/T₁ and 1/T₂ are proportional to absolute O₂ concentration; (3) Robust data fitting model. With the decrease in SNR, pulse EPR O₂ precision does not fall as rapidly as for CW due to a much simpler fitting model (mono-exponential function for T₂ or T₁ measurements vs multi-parametric CW linewidth fit). Very few measurement points, as low as 10, are sufficient for relaxation time determination; (4) Simplicity of software. The inventors developed automatic and semi-automatic acquisition and processing software for pulse EPR data where no user input or special knowledge is required; and (5) Pulse acquisition offers an order of magnitude faster and more accurate O₂ images. Pulse acquisition is very fast (down to millisecond per point) and thus less sensitive to motion and ambient noise.

With respect to the choice of frequency, the EPR signal-to-noise ratio grows with the frequency while an increase in frequency demands higher RF power and reduces the penetration depth of RF into samples with high conductivity and dielectric losses. These trends produce an optimal measurement frequency, different for each experimental circumstance. For example, for human volume imaging most magnetic resonance instruments are below 400 MHZ, while for small rodents, frequencies in the range of 600 MHZ-1200 MHZ are used. For non-conductive, and low-loss samples, frequencies above 9 GHz are common. In the present case, the RFMC resonator environment is only in part formed by the lossy tissues outside of the needle, while the LiPc probe and needle materials have negligible losses. Therefore, an optimum frequency above 1 GHz is anticipated.

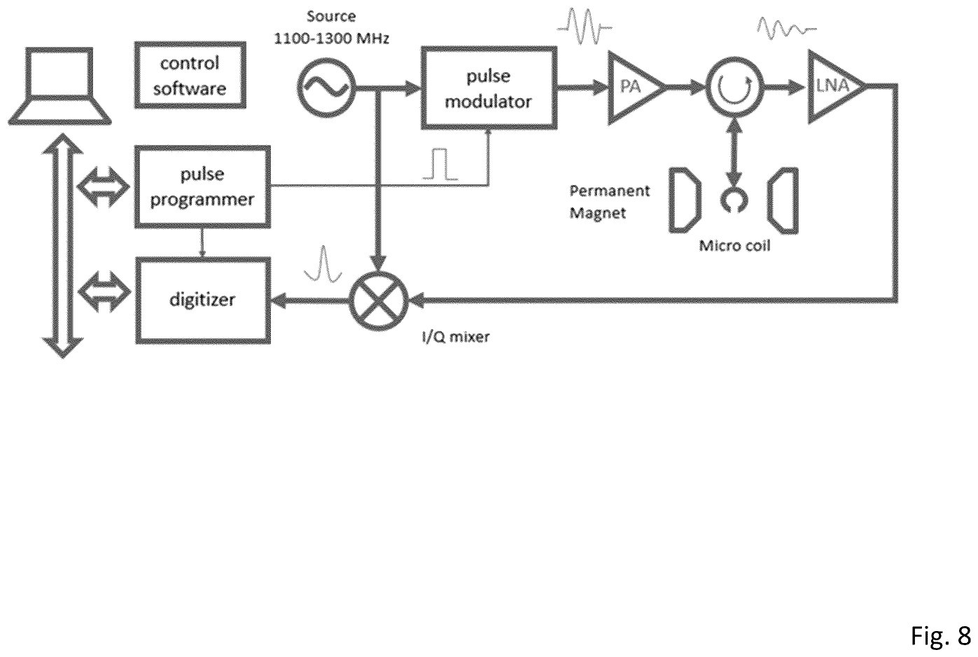

Regarding the pulse EPR instrument, an L-band magnet system with gradients (41 mT with 80 mT/m maximum gradient strength) is the highest field human-size EPR magnet readily available. The system will also include an L-band homodyne bridge. FIG. 8 is a simplified diagram of the L-band system for use in the proposed system in accordance with an illustrative embodiment. The pulse modulator of the bridge will enable 90-degree step phase cycling and pulse amplitude modulation (e.g., 0-7.5 dB). All components of the bridge are commercially available or readily manufacturable. As an example, key components of the bridge can include an SynthNV RF source, a FEMIPULSE FPGA pulse programmer, a PN1004 EPR power and phase modulator, a PE15A4025 2W power amplifier, an SC5312C mixer, a M4i.2211 digitizer, and SpecMan4EPR control software. In alternative embodiments, different system components may be used.

The RF technology is well established. While T₂ measurement sequences are simpler and deliver less power to the sample, T₁ measurement has, in some cases, demonstrated additional advantages. Thus, the inventors will test both methods and apply the best. For T₁ determination the inventors will use inversion recovery electron spin echo sequence (IRESE) and saturation by repetition time (SRT) sequences.

Discussed below is the development of an RFMC that can be fed through HDR tubes and needles and used for LiPc-based oximetry. One unique aspect of the RFMC is that the sensor is located, and the magnetic field B₁ is maximized, outside the coil. The outer dimensions of the RF coil are limited by the inner diameter of the brachytherapy needle of ˜0.9 mm while the LiPc probe is located ˜0.5-1 mm from the needle axis (see insert of FIG. 7). The art of designing an RFMC suitable for the application involves maximizing the magnetic field inside the LiPc probe (located on the outer surface of a specially designed HDR needle) while maintaining most of the electric fields within the needle. This can be achieved by proper choice of the needle material and coil geometry.

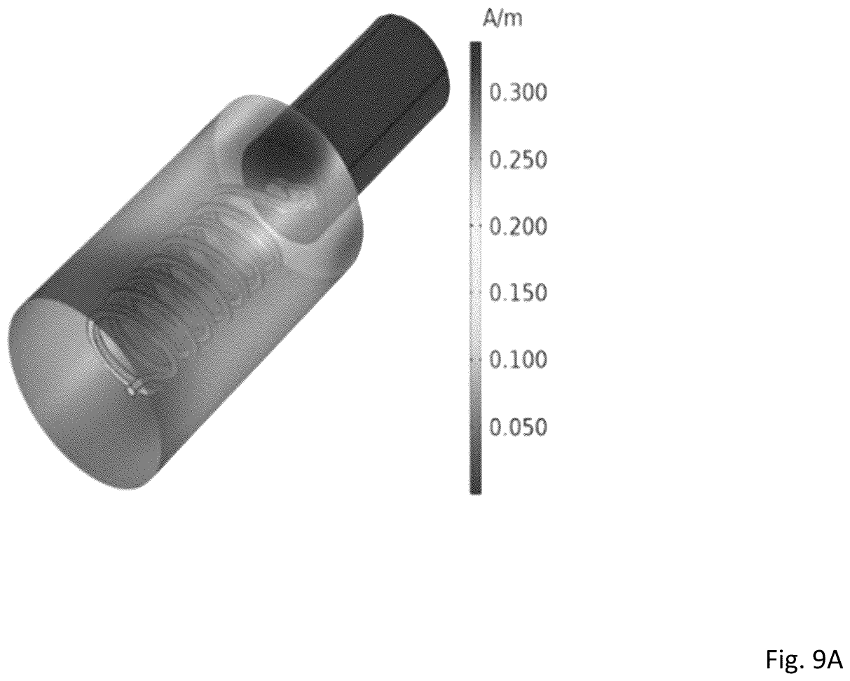

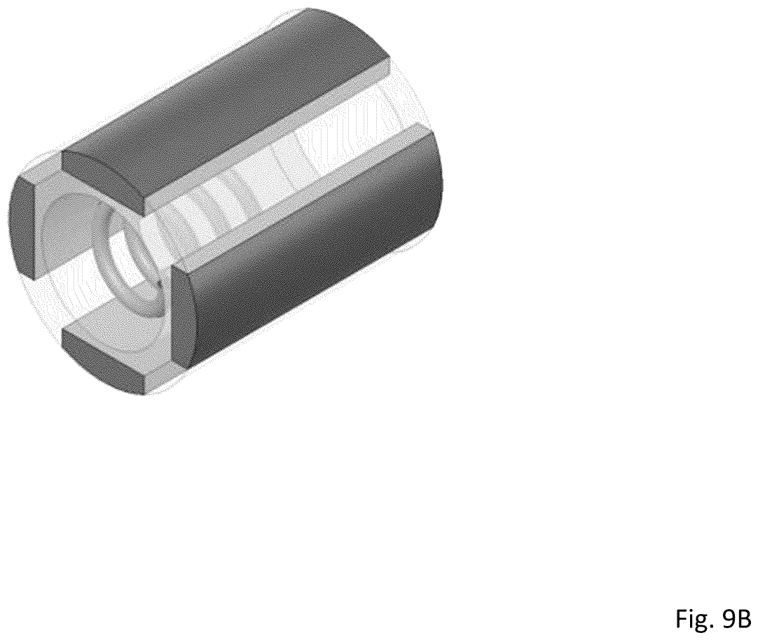

Design of the RFMC will be optimized and verified by electromagnetic simulations using finite element analysis software (e.g., COMSOL Multiphysics®). For each RFMC design, parametric sweep simulations are conducted while allowing orientation, size, and materials of RFMC components to vary within the boundaries of the HDR needle ID seeking to maximize: 1) the ability to produce a strong, B₁⊥ field around the O₂-sensitive LiPc layers on the outer HDR needle walls, 2) the power-to-field conversion efficiency at the sample position, and 3) the antenna's directionality. FIG. 9A shows B₁⊥ simulations of a slant helix coil formed at the end of a transmission line conducted using the COMSOL Multiphysics® analysis software in accordance with an illustrative embodiment. The RFMC geometry was defined using parametric equations to allow parameter sweep studies varying in nearly all dimensions. FIG. 9B is a rendering of a radio frequency micro-coil in accordance with an illustrative embodiment. As shown, the RFMC (coil) is positioned within a cut-off section of an HDR needle that has grooves (grey) filled with LiPc.

To simulate the O₂-sensitive region, a 3 mm long, 0.1 mm thick cylindrical layer with an outer diameter matching that of the HDR needles (1.98 mm) was modeled and used as the region of interest. An input RF power of 1 W (1.5 GHZ) and an impedance of 50Ω was set in all simulations. Among the designs analyzed thus far, the slant helix provides strongest fields (FIG. 6) at the probe position, exceeding the initial performance requirements and will thus be optimized for imaging in the proposed system.

System design also includes determining preferential spin probe placement and adapting coil design to maximize magnetic fields at the position of the probe. To determine the preferential placement of LiPc-coated regions the inventors calculate, and plot normalized

$${B}_{2}^{1} ⊥$$

values for each RFMC design along the x, y, and z coordinates, and an estimate of the active volume is obtained by measuring the distance between the boundaries where

$${B}_{2}^{1} ⊥$$

drops significantly (by ˜80%). The trends in simulations of field distributions can be verified by comparing the loss in EPR signal in experiments or in

$${B}_{2}^{1} ⊥$$

in simulations when the LiPc sample is shifted from the active region along x, y, and z individually. In the case of resonant structures, reflected power plots will be computed and compared against network analyzer measurements to determine agreement between the simulated and measured resonant frequency and Q-factor values.

The inventors also ensured that the resonator coupling unit is compatible with the autosequencer. Coupling of a resonator is a straightforward task. The purpose of the coupling unit is to transform coil impedance to 50 Ohm to allow effective signal transmission. The system can use a capacitive step-up coupling. However, the placement of the coupling unit next to the coil may be difficult due to spatial restrictions. Thus, RFMC coupling can be located at the end of the transmission line. Initial tests demonstrated that coupling exceeding 40 dB can be achieved with a 30 cm transmission cable (the cable from the coupling unit can be of any length). Matching capacitors will be adjusted at conditions resembling the tissues and will be non-tunable. The bridge control will automatically adjust the EPR frequency and field to match the resonator frequency.

Potential pitfalls and their resolution are discussed below. Ideally, a uniform B₁⊥ in the sample should be created. As can be seen in FIG. 6, the slant helix micro-coil provides incomplete coverage due to field orientation (a single loop shows similar performance). If this presents a problem, the system may use a coplanar microstrip coil design, common for many EPR-on-a-chip applications, or simple solenoidal coils. Although it is expected that resonant structures have higher sensitivity attributable to the high-quality factor (Q-factor), it has been found that some non-resonant structures achieve comparable sensitivity due to an increase in B₁⊥ field homogeneity over the sample volume, and are potentially advantageous in pulse EPR due to their broadband nature. If coil coupling is significantly affected by its position within the body, the adjustment of coupling may be performed. In this case, one can use a digital remote matching unit. For signal enhancement, the RFMC may be wound on high dielectric constant core.

As discussed herein, establishing sparse 3D oxygen imaging using the RFMC is a primary goal of the system. The O₂ distribution in tumors is highly heterogeneous (see FIG. 2). Described below is a 3D O₂ imaging protocol for HDR needles. The main imaging approach is the mechanical movement of the RFMC by the autosequencer along the needle channels within the patient. Because the coil sensitivity will not exceed more than a few millimeters beyond the coil, the acquired O₂ data can be directly mapped to this region thus forming a sparse 3D image. The Varian autoloader frame of coordinates can be used to assemble the final image from the multitude of measurements performed in many positions along each needle. In this frame, O₂ data will be directly mapped into the treatment planning software.

With respect to the acquisition methodology, T₂-based oximetry and electron spin echo (ESE) pulse sequence that provide the highest SNR and lowest sensitivity to the coil fringe fields and magnetic field inhomogeneity can be used. Relaxation times of LiPc are within 300 nanoseconds (ns) to 5 microseconds (μs) (21% to 0% O₂), thus a repetition time TR of 50 μs can be used in one embodiment. The data produced with 16-step phase cycling and 10 points along relaxation domain can be averaged 60 times to boost SNR, resulting in ˜0.5 s per acquisition with high SNR.

Considering the sensitivity pattern as observed in FIG. 6, four independent measurements can be generated at each RFMC position (e.g., ‘top’, ‘bottom’, ‘left’, ‘right’) from only 3 projections (only 1.5 seconds). Reconstruction can be achieved using Directed Total Variation image reconstruction in conjunction with signal modeling based on geometrical constraints. A gradient of 10 mT/m can be used to resolve the spatial positions separated by 2 mm.

Because the applied gradient will alter the average magnetic field at the spin probe position, the EPR signal frequencies will change (the basis of EPR imaging). For large objects, this change can exceed the resonator bandwidth. To maintain acquired signals within the resonator bandwidth a countering field offset can be applied. The value of the offset can be calculated from the geometrical model of needles. Reciprocally, knowing the countering offset and EPR signal frequency offset under the gradient allows one to precisely map signals to the spatial positions in the magnet.

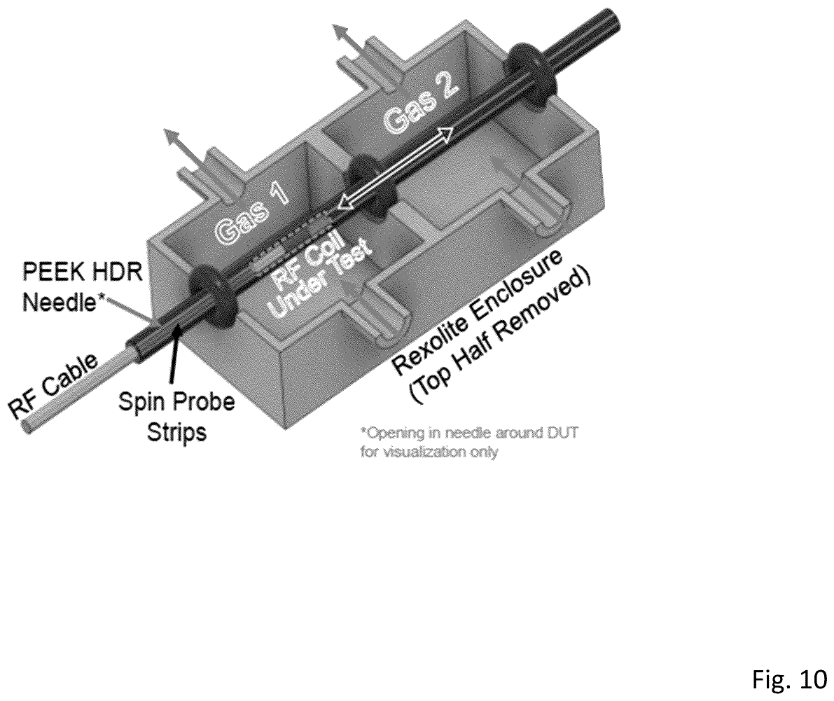

Validation of RFMC sparse 3D imaging with L-band pulse bridge can be performed using a test setup. The test setup for evaluation can include the needle mockup with an embedded LiPc probe placed into an enclosure separated into 2 or 4 chambers to simulate the heterogeneous environment. FIG. 10 depicts the layout of a test chamber for validating the RFMC in accordance with an illustrative embodiment. The test chamber utilizes 2 gases in two sub-chambers, and the RFMC is inserted into a central channel of the test chamber and can move between the two sub-chambers. The test setup can be placed at the center of the L-band system. The test chamber can be constructed using gas-impermeable plastic (e.g., rexolite) in one embodiment.

For coil performance evaluation, the chambers of the test system can be filled with N₂. The test protocol can include recording i) the amplitude and SNR of the EPR signal, ii) optimum power, and iii) instrument dead time for each coil. The coil with the highest amplitude and SNR, lowest optimum power, and shortest dead time can then be selected. For imaging performance evaluation, the chambers of the test system can be filled with N₂ and 3% O₂ gasses respectively. The coil can be moved through the enclosure to observe the transition in measured O₂. Axial resolution can then be determined. The same setup can be used to evaluate a benchtop autosequencer and L-band control software. For 2D in-plane imaging tests, a setup with isolated vertical sub-chambers may be used. This enables determination of the ability of the system to resolve O₂ on the ‘upper’ and ‘lower’ portions of the needle.

The phantom test setup of FIG. 10 can be used to validate the performance of the EPR-compatible HDR needles and RFMC. The enclosure can be constructed from a sheet of gas-impermeable Rexolite plastic with multiple chambers that can be filled with a variety of gas mixtures. Chambers will have gas inlets and outlets with luer connectors. Each chamber can be filled independently. A channel for the HDR needle can be cut through the chambers and isolated with rubber O-rings for an airtight seal. A sheet of Rexolite can then be glued over the top of the chambers ensuring an airtight seal. The phantom will be compatible with both gas and liquid media.

For characterization of the brachytherapy EPR O₂ imaging needle and L-band pulse EPR system, additional tests can be performed using the phantom. These additional tests can include: i) Precision and accuracy of pO2 measurements. The RFMC will be positioned in the middle of the phantom chamber and a series of measurements will be taken for 0%, 3%, and 6% O₂ gas mixtures. The standard deviation and systematic error can be calculated at each gas setting; ii) Time per measurement necessary to achieve a standard deviation of 2 torr (i.e., determine the average time necessary to achieve less than 2 torr standard deviation for 0%, 3%, and 6% O₂ gas mixtures); iii) Spatial image resolution in axial direction. The phantom can be filled with 0%, 3%, and 6% O₂ gas mixtures in different chambers. The RFMC will be driven through the needle to observe the change of O₂ in the axial needle direction. The movement of the coil required for changing O₂ by 80% of the expected difference will be considered as an image resolution; iv) The response time of the spin probe to changes in O₂ concentration (i.e., select one chamber and alternate the gas inside from 0 to 3% and measure the reaction time of the spin probe).

For verification of the electric characteristics of the RFMC such as coupling, Q-factor, and optimal power, the phantom can be used with chambers filled with a liquid that mimics tissue dielectric properties. This will allow one to see the RFMC performance in a lossy environment resembling human tissue. The phantom media can follow a standard recipe that includes distilled water, salt, oil, dishwashing detergent, and a solidifying agent.

Additional testing of the HDR needles suitable for EPR imaging can also be performed. In one embodiment, the system can use LiPc O2-sensitive spin probe, which is a water-insoluble crystalline powder. LiPc has extremely narrow linewidth at 0% O₂, very high spin density (about 8×10¹⁹ spins/cm3) and high O₂ sensitivity (˜7 mG/torr124). Both spin-spin (R₂=1/T₂) and spin-lattice (R₁=1/T₁) relaxation rates of the probe are proportional to the O₂ concentration, which increases the relaxation rate of the probe via the Heisenberg spin exchange between the spin probe and the unpaired electrons of molecular O₂.

The LiPc produced with the scalable method discussed herein can be fully characterized for clinically relevant parameters such as purity, O₂ relaxation rate sensitivity, signal stability in the context of radiation, and high temperatures used for sterilization. Other aspects like probe leaching from the needle to surrounding tissues can be characterized during validation.

A full characterization of LiPc can be performed by producing batches of LiPc and determining the variation in XRD structure and pulse EPR signal generated by 5 mg of the sample under N₂ atmosphere. The absolute spin density of the probe can be calculated using a reference LiPc sample calibrated according to the standard procedure in relation to DPPH radical. The O₂ concentration dependence of R₂=1/T₂ and R₁=1/T₁ can also be determined. For the test, a standard 5 mm loop-gap resonator can be used. Samples can be placed in a 5 mm diameter tube covered with a membrane penetrated by needles circulating gas mixtures with 0, 3%, and 6% O₂.

The stability of the LiPc signals and O₂ sensitivity are then tested at three clinically relevant conditions: (a) all HDR needles come sterilized for clinical use, as such the LiPc is subjected to autoclave sterilization cycles (e.g., 121° Celsius for 30 min, 25 cycles); (b) radiation test at 5 Gy, 30 Gy and 125 Gy representing single fraction and typical HDR prescription doses (in cervical cancers), and a 25 fraction dose respectively; and (c) shelf life by storing in sealed packaging inside a desiccator vacuum with testing every 30 days over a year. The probe's stability can be determined by comparing EPR signals and XRD structures before and after each test. In one embodiment, a test pass condition can be an absolute pO₂ accuracy of 3 torr, with reproducibility of 2 torr. It is noted that an alternative spin probe such as LiNc-BuO⁸ can also be considered.

To achieve an EPR-compatible needle, O₂-sensitive material must be incorporated into the outer layer of the HDR needles. The needle design requirements can include being made from bio-compatible material, being REACH/RoHS compliant, having the LiPc coating be robustly attached so that it does not flake/detach, having a durability of at least 25 sterilization/reprocessing cycles, having a size that is compatible with existing templates, being MRI compatible, and also ensuring that the LiPc distribution works with the EPR coil radiation platform. The insert of FIG. 7 shows an example design of the needle.

The first approach to implement this design is to machine a set of grooves in a standard needle (in production these will be formed when the needle is extruded with a custom die) and then fill grooves with a LiPc and PDMS mixture using co-extrusion through an orifice with a diameter equal to needle diameter. The solidified PDMS will take the shape of the needle and mechanically hold the LiPc in place. The advantage of this approach is the well-characterized PDMS-LiPc system with high O₂ permeability. Material biocompatibility and REACH/RoHS can be addressed through working with medical grade materials.

The LiPc coating can be tested by (a) repeated insertion and extraction of the final prototypes in a tissue-mimicking material; (b) flexing while inserted in a tissue-mimicking material; (c) subjecting needles to 30 Gy and 125 Gy radiation; and (d) subjecting needles to sterilization cycles. The remnants after completion of tests will be analyzed with EPR for any trace of LiPc material that may have detached from the needle. The in vivo stability of LiPc signals is less of a concern since the needles remain in the patient for a maximum of 3 days and LiPc probes with PDMS coating were successfully validated in rabbits for up to 14 days. The result is a robust HDR needle impregnated with LiPc spin probe design tested for signal stability under high temperature and irradiation conditions.

Alternative implementations can include 1) direct incorporation of the LiPc into the plastic material of the needle, a robust approach that may be limited by O₂ diffusion and LiPc temperature stability; (2) uniform coating of LiPc over the needle surface with a top conformal coating (e.g. silicone) for material retention and mechanical integrity. Advantages are positionally and directionally independent distribution of LiPc and simple application, but a thin coating may limit the density of material (i.e., signal strength) and may be fragile; (3) using spiral grooves filled with PDMS-LiPc; and (4) using alternatives to PDMS.

An autosequencer was also developed and adapted to RFMC for automatic mechanical positioning within HDR needles to achieve O₂ imaging. For HDR treatment, an afterloader automatically sequences the radioactive seed through multiple needles to specific locations. To take advantage of this technology, the proposed sensor system is mechanically compatible with the current VMS afterloader. To facilitate the development, a benchtop autosequencer resembling the existing Bravos HDR Varian autoloader can be built for the EPR probe. In one embodiment, it can feature the same HDR needle connection mechanism used in the clinical Bravos system.

In one embodiment, the radioisotope afterloader has two separate channels, one with a dummy wire to perform QA checks and the other carries the source itself. The benchtop design can be simplified to a single channel, only carrying the RFMC itself. A prototype concept illustrating some of the critical modules from the clinical device is shown in FIG. 7. The benchtop autosequencer controller can be developed to provide direct integration with L-band control software via serial over USB or TCP/IP command. This will allow automated tests of 3D imaging.

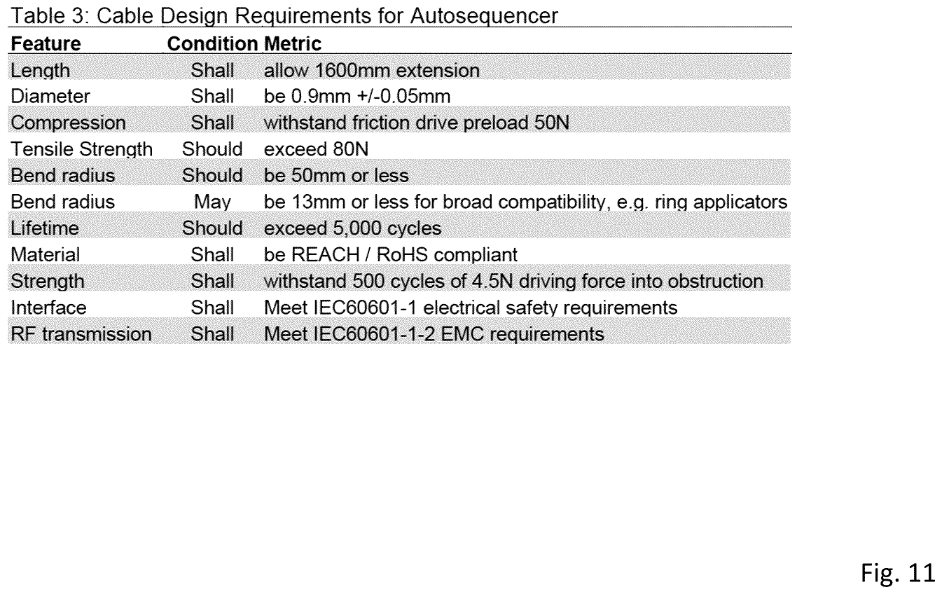

Mechanical and electric coupling of the RFMC to the autosequencer and L-band bridge is one of the engineering challenges of this system. The cable should serve both as a transmission line for the RF signals and guide wire for the autosequencer. FIG. 11 is a table that depicts cable design requirements for the autosequencer in accordance with an illustrative embodiment. In alternative embodiments, different metrics may be used. The cable should be stiff enough to allow precise position manipulation yet flexible to allow coiling and following the path of the transfer guide tubes and needles. From the electrical standpoint, the cable should have minimum losses and maintain impedance upon bending. Preliminary results using UT-047 coaxial cable (1.2 mm OD, 0.4 dB/ft attenuation) show that 37 cm long coaxial transmission line coupled with a 7-turn copper RFMC (resistivity 1.68 μΩ-cm) enable CW EPR O₂ measurements with an SNR of 280 for a single 5-second scan and is a good candidate for this purpose.

In an illustrative embodiment, any of the operations described herein can be performed by a computing device that includes a processor, memory, user interface, transceiver (e.g., a transmitter and a receiver), etc. For example, the operations can be stored as computer-readable instructions in the memory, and the processor can execute the computer-readable instructions to perform the operations described herein.

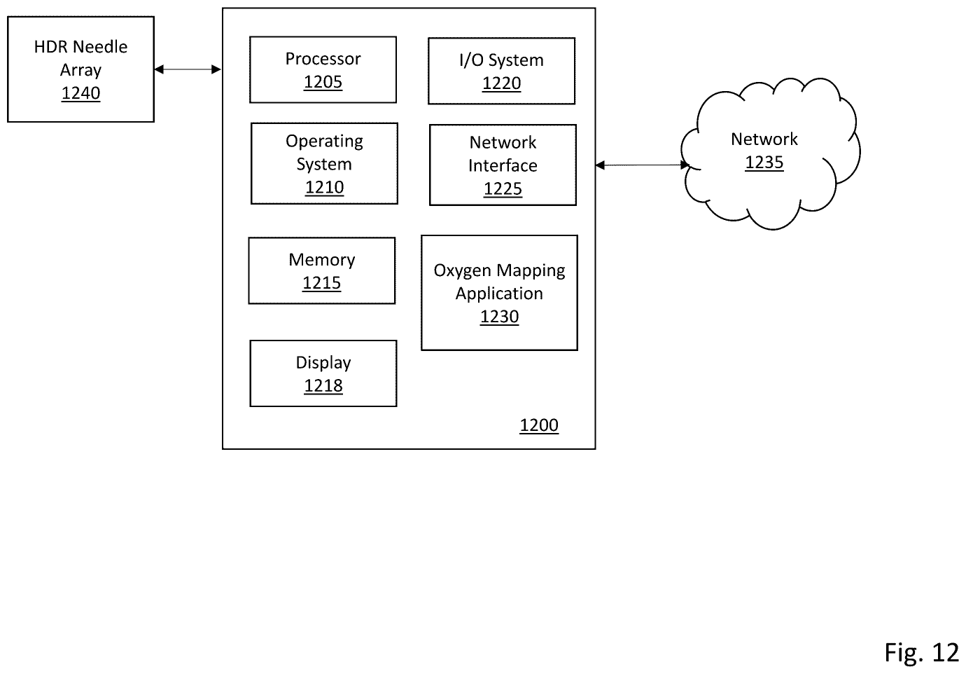

FIG. 12 depicts a computing system 1200 for measuring and mapping oxygen levels of tumors in accordance with an illustrative embodiment. In one embodiment, at least a portion of the computing system 1200 can be part of an autosequencer that is connected to and controls an HDR needle array 1240. Alternatively, the computing system 1200 may be remote from the HDR needle array 1240, but in communication therewith. The computing system 1200 can communicate with other devices, databases, websites, etc. through a network 1235 or other form of wireless communication.

The computing system 1200 includes a processor 1205, an operating system 1210, a memory 1215, a display 1218, an input/output (I/O) system 1220, a network interface 1225, and an oxygen mapping application 1230. In alternative embodiments, the computing system 1200 may include fewer, additional, and/or different components. The components of the computing system 1200 communicate with one another via one or more buses or any other interconnect system. The computing system 1200 can be part of an autosequencer, part of an imaging system, a standalone device connected to the autosequencer or imaging system, or any other type of computing system (e.g., smartphone, tablet, laptop, desktop, etc.), including a dedicated standalone computing system that is designed to perform the tumor oxygen level mapping.

The processor 1205 can be in electrical communication with and used to control any of the system components described herein. For example, the processor 1205 can be used to execute the oxygen mapping application 1230, control the HDR needle array 1240, process image data received via the HDR needle array 1240, control delivery of brachytherapy, control the display 1218, present oxygen maps on the display 1218, etc. The processor 1205 can be any type of computer processor known in the art and can include a plurality of processors and/or a plurality of processing cores. The processor 1205 can include a controller, a microcontroller, an audio processor, a graphics processing unit, a hardware accelerator, a digital signal processor, etc. Additionally, the processor 1205 may be implemented as a complex instruction set computer processor, a reduced instruction set computer processor, an x86 instruction set computer processor, etc. The processor 1205 is used to run the operating system 1210, which can be any type of operating system.

The operating system 1210 is stored in the memory 1215, which is also used to store programs, received image data, received needle data, network and communications data, peripheral component data, the oxygen mapping application 1230, and other operating instructions. The memory 1215 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 1215 can be in the cloud to provide cloud storage for the system. Similarly, in one embodiment, any of the computing components described herein (e.g., the processor 1205, etc.) can be implemented in the cloud such that the system can be run and controlled through cloud computing.

The I/O system 1220 is the framework which enables users and peripheral devices to interact with the computing system 1200. The display 1218 can include a touch screen in some embodiments, and the touch screen can be part of the I/O system 1220 that allows a user to make selections, control sub-systems, view results, etc. The display 1218 can be any type of display, including a monitor, projector, etc., and can be used to present user interface screens, control screens, captured images, captured video, generated maps, and other data to the user. The I/O system 1220 can also include one or more speakers, one or more microphones, a keyboard, a mouse, one or more buttons or other controls, etc. that allow the user to interact with and control the computing system 1200. The I/O system 1220 also includes circuitry and a bus structure to interface with peripheral computing devices such as an imaging system, sensors, power sources, universal service bus (USB) devices, data acquisition cards, peripheral component interconnect express (PCIe) devices, serial advanced technology attachment (SATA) devices, high-definition multimedia interface (HDMI) devices, proprietary connection devices, etc.

The network interface 1225 includes transceiver circuitry (e.g., a transmitter and a receiver) that allows the computing system 1200 to transmit and receive data to/from other devices such as remote computing systems, servers, websites, imaging systems, etc. The network interface 1225 enables communication through the network 1235, which can be one or more communication networks. The network 1235 can include a cable network, a fiber network, a cellular network, a wi-fi network, a landline telephone network, a microwave network, a satellite network, etc. The network interface 1225 also includes circuitry to allow device-to-device communication such as Bluetooth® communication.

The oxygen mapping application 1230 can include software and algorithms in the form of computer-readable instructions which, upon execution by the processor 1205, performs any of the various operations described herein such as controlling the HDR needle array 1240, generating images based on data received from the HDR needle array 1240, controlling movement of RFMC sensors within needles of the array, controlling the autosequencer, controlling the EPR bridge, presenting generated images on the display 1218, etc. The oxygen mapping application 1230 can utilize the processor 1205 and/or the memory 1215 and/or the display 1218 as discussed above. In an alternative implementation, the oxygen mapping application 1230 can be remote or independent from the computing device 1200, but in communication therewith.

Thus, described herein is a first-in-class, non-invasive clinical sparse 3D O₂ imaging technique that provides direct, accurate, and fast serial O₂ measurements. With its high temporal resolution, it can track tumor oxygenation dynamics, which is crucial for successful implementation of personalized treatment strategies such as oxygen-guided HDR brachytherapy. The system utilizes oxygen sensors incorporated into needles used to deliver brachytherapy. This lets one measure oxygen levels quickly and directly, without adding any extra invasiveness or discomfort for patients. This integration of HDR with EPR oximetry not only addresses key existing limitations but also opens new possibilities for volumetric oximetry. By leveraging multiple HDR needles (˜20) already placed in the tumor for brachytherapy, this approach will, for the first time, enable doctors to (1) target higher RT doses more precisely to resistant tumor areas, 2) identify which patients benefit from O₂-enhancing intervention and 3) time treatment when it is most effective. The technological advancements present a pivotal leap from single-point EPR oximetry to clinically relevant volumetric O₂ mapping, addressing a key barrier to the clinical translation of personalized treatments.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

1. A system for mapping oxygen levels in tumors, comprising:
a needle that is sized to deliver high dose rate brachytherapy to tissue into which the needle is inserted, wherein the needle includes a radio frequency micro-coil sensor positioned within a shaft of the needle and a spin probe mounted to an outer surface of the shaft of the needle; and
a processor in communication with the needle, wherein the processor is configured to:
move the radio frequency micro-coil sensor along the shaft of the needle;
receive, from the radio frequency micro-coil sensor, oxygen level readings at a plurality of tissue locations along the shaft of the needle; and
generate an oxygen level mapping of the tissue based on the oxygen level readings received from the radio frequency micro-coil sensor.

2. The system of claim 1, further comprising a radio frequency cable connected to the radio frequency micro-coil sensor and used to move the radio frequency micro-coil sensor along the shaft of the needle.

3. The system of claim 1, wherein the spin probe comprises a strip of lithium phthalocyanine (LiPc) mounted to the outer surface of the shaft of the needle.

4. The system of claim 3, wherein the needle includes a plurality of strips of LiPc that are mounted lengthwise along the outer surface of the shaft of the needle.

5. The system of claim 1, wherein the processor is configured to determine a dosage of treatment at each location of the plurality of tissue locations based on the oxygen level reading at each location.

6. The system of claim 5, wherein the processor is configured to deliver the dosages of treatment through the needle.

7. The system of claim 5, wherein the dosage of treatment corresponds to the oxygen level at a given tissue location such that the dosage of treatment increases as the oxygen level decreases.

8. The system of claim 1, further comprising a layer of biocompatible oxygen permeable material that encapsulates the spin probe.

9. The system of claim 8, wherein the layer of biocompatible oxygen permeable material comprises polydimethylsiloxane (PDMS).

10. The system of claim 1, wherein the processor uses an L-band pulse electron paramagnetic resonance (EPR) imaging system to generate the oxygen level mapping.

11. The system of claim 1, wherein the radio frequency micro-coil sensor comprises a dielectric core and a slant helix coil mounted on the dielectric core.

12. A method for mapping oxygen levels in tumors, comprising:
moving, by way of a radio frequency cable, a radio frequency micro-coil sensor along an interior of a shaft of a needle that is sized to deliver high dose rate brachytherapy to tissue into which the needle is inserted;
receiving, by a processor in communication with the radio frequency micro-coil sensor, oxygen level readings at a plurality of tissue locations along the shaft of the needle; and
generating, by the processor, an oxygen level mapping of the tissue based on the oxygen level readings received from the radio frequency micro-coil sensor.

13. The method of claim 12, wherein the needle includes a spin probe mounted to an outer surface of the shaft of the needle.

14. The method of claim 12, wherein the spin probe comprises a strip of lithium phthalocyanine (LiPc) mounted to the outer surface of the shaft of the needle.

15. The method of claim 12, further comprising determining, by the processor, a dosage of treatment at each location of the plurality of tissue locations based on the oxygen level reading at each location.

16. The method of claim 15, further comprising delivering, by the processor, the dosages of treatment through the needle.

17. The method of claim 15, wherein the dosage of treatment corresponds to the oxygen level at a given tissue location such that the dosage of treatment increases as the oxygen level decreases.

18. The method of claim 12, wherein a layer of biocompatible oxygen permeable material encapsulates the spin probe.

19. The method of claim 12, further comprising using, by the processor, an L-band pulse electron paramagnetic resonance (EPR) imaging system to generate the oxygen level mapping.

20. The method of claim 12, wherein the radio frequency micro-coil sensor comprises a dielectric core and a slant helix coil mounted on the dielectric core.
